(19) United States
(12) Patent Application Publication
(10) Pub. No.: US 2013/0287812 A1
(43) Pub. Date: Oct. 31, 2013

(54) anti-HPV drug and its application in preparing medicines for treatment of HPV (human papilloma virus) infection

(71) Applicants: Jun Gao, Liaoning (CN); Weicheng Zhao, Liaoning (CN); Zhumu Bai, Liaoning (CN); Huilin Zhao, Liaoning (CN); Qingyi Zhang, Liaoning (CN); Haichun Yu, Liaoning (CN); Junwei Yang, Liaoning (CN); Feifei Sun, Liaoning (CN)

(72) Inventors: Jun Gao, Liaoning (CN); Weicheng Zhao, Liaoning (CN); Zhumu Bai, Liaoning (CN); Huilin Zhao, Liaoning (CN); Qingyi Zhang, Liaoning (CN); Haichun Yu, Liaoning (CN); Junwei Yang, Liaoning (CN); Feifei Sun, Liaoning (CN)

(21) Appl. No.: 13/504,210

(22) PCT Filed: Nov. 11, 2011
(86) PCT No.: PCT/CN11/82084

§ 371 Date: Apr. 26, 2012

(30) Foreign Application Priority Data
Jan. 18, 2011 (CN) 201110021347.0

(51) Int. Cl. A61K 39/205

(52) U.S. Cl. CPC A61K 39/205
USPC 424/224.1

(57) ABSTRACT

This invention disclosed an anti-HPV drug and its applications in preparing medicines for treatment of HPV (human papilloma virus) infection. This drug was characterized by using human rabies vaccine as its active ingredient and being produced by adding pharmacologically acceptable carriers. This invention also disclosed drug applications in preparing medicines for treatment of HPV infection, i.e. new applications of human rabies vaccines for preparing medicines against HPV infection. The anti-HPV drug described in this invention (i.e. human rabies vaccine) had obvious treatment effects on HPV infection. According to the animal tests and preclinical observation, this drug provided satisfactory and beneficial effects and had the characteristics of safety, effectiveness and nearly no recurrence after treatment; the negative-conversion rate in patients with positive HPV-infection reached 90% after treatment.

### TECHNICAL FIELD

The invention relates to a biological product, in particular to an anti-HPV drug containing human rabies vaccines and the application of the human rabies vaccine in preparation of the medicines for treatment of the HPV infection, which belong to the bio-pharmaceutical technology.

### BACKGROUND OF THE INVENTION

Human papilloma virus (HPV) is a virus from the papillomavirus family. It is a small, spherical, double-stranded, circular DNA-containing virus without an envelope. At present, more than 120 types of HPV have been found, capable of being divided into high-risk type or low-risk type in terms of carcinogenicity. An epidemiological classification research defined 15 types of high-risk HPVs (such as HPV-16 and HPV-18) and 12 types of low-risk HPVs (such as HPV-6 and HPV-11).

HPVs are common viruses widely transmitted among people. Most HPV infections cause no symptoms in people and are self-limited. The existing epidemiological data shows that people are typically susceptible to many types of HPVs, and sexually active young people are considered to be at a high risk of HPV infection. Patients and latently-infected HPV carriers are the major source of infection, and HPV is mainly transmitted through close contact, typically sexual contact. Recently, HPV inflection has increased considerably, in particular among the sexually active young people. An American study made in 2006 showed that there were 20 millions of HPV infectors, 0.62 millions of them newly infected (Trottier H, Franco E L. Human papillomavirus and cervical cancer: Burden of illness and basis for prevention. Am J Manag Care, 2006, 12(17 suppl):S462-S472). Estimated from the epidemiological researches, 74% of the new HPV infectors ages from 14-24 each year, the risk that pregnant women are infected by HPV viruses is increased and that the immunity inhibitor and human immunity virus (HIV) infectors infecting HPV and generating related pathological changes increases. HPVs establish productive infection in the skin and mucous membranes by self-replication in the host's nucleuses or integration in the DNA of the host's cells and synchronous replication and transcription along with the host's cells. The virus replication causes epitheliosis to make the epidermis thicken and generate acanthosis and hyperkeratosis, and then at last leads to epitheliosis to form papilloma, also called warts. Generally, the warts are benign, in which DNA of the PHV is free. However, some types of HPVs have the abilities of inducing cellular transformation, and the DNA thereof can be integrated in the chromosomes of the host's mucous membranes to promote synthesis of DNA of the cells and the skin. In particular, the mucous membranes change into immortal cells, so as to cause precancerous lesions or malignant tumors.

HPV infections are capable of causing many types of pathological changes which are diversified in clinic symptoms, usually causing severe diseases, such as cervicitis, cervical erosion, skin papilloma, genital wart, etc., even malignant tumor, to pregnant women and people with low immunities.

At present, most positive HPV infections are treated by external therapies, and some by internal therapies. There are many external therapies, such as pharmacotherapy, cold therapy, laser therapy, microwave therapy, electrocauterization and surgical therapy. The pharmacotherapy is convenient and easy to operate, but some drugs may burn the mucous membranes. Injection of immune potentiators such as interferon and interleukin, which are expensive, has some effects that are still under discussion in the academic circles, and most people will suffer from certain side effects. None of the mentioned therapeutic solutions can avoid recurrence of the HPV infection.

Human rabies vaccines are rabies preventive preparations prepared by the steps of cultivating and augmenting rabies viruses, collecting and inactivating the virus liquid, and then properly purifying the obtained products, wherein the human rabies vaccines can be produced from different rabies strains, such as PV-2061, PM, Vnukovo-32, Flury, aG, CTN-1, CVS, etc. Besides, other effects of the vaccines draw much attention. At the beginning of the last century, articles about treating cervical cancer with “rabies” were published in the American Journal of Medicine in 1912. From the '90s to the beginning of this century, articles about treating tumors with viruses were published successively. (The article Annals of Surgical Oncology. 10(6):596-605, New Approaches to the Treatment of Hepatic Malignancies Viral Oncolysis for Malignant Liver Tumors issued in July of 2003 also had a description on the anti-tumor effects of rabies viruses, adenoviruses, herpes simplex viruses, etc., all of which are called oncolytic viruses).

The Chinese patent application CN1778389 “Medicine for treating tumor and its application in preparation of medicine for treating tumor” discloses the application of a rabies vaccine in tumor treatment, in particular to a tumor therapeutic drug using the rabies vaccine as a single ingredient and the application thereof in preparation of other tumor therapeutic drugs.

The Chinese patent application CN101524539 “New application of rabies human immunoglobulin in preparing medicine for preventing and controlling Japanese encephalitis and combined vaccine for rabies and Japanese encephalitis” discloses the new application of the human rabies immunoglobulin in preparation of drugs for preventing and treating Japanese encephalitis. However, the report about using the human rabies vaccine to treat HPV infection has not been found yet.

### CONTENT OF THE INVENTION

One purpose of this invention is to provide a new drug for treatment of HPV infection. The active ingredient of this drug is the human rabies vaccine, which has good curative effects on HPV infection and no side effects or toxic effects.

Another purpose of this invention is to provide the application of this drug in preparing medicines for treatment of the HPV infection, that is the new application of the human rabies vaccine in the preparation of medicines against HPV infection.

The invention supplies the technical solution that: the anti-HPV drug is characterized by using human rabies vaccine as its active ingredient and being produced by adding pharmacologically-acceptable carriers. To better realize the purposes of this invention, the human rabies vaccine can be prepared from any one of following rabies strains: PV-2061 strains, PM strains, Vnukovo-32 strains, Flury strains, aG strains, CTN-1 strains and CVS strains.

To better realize the purposes of this invention, the anti-HPV drug may be administered by injection, spray, gel or suppository, wherein each dose contains 0.1-15 IU of the human rabies vaccine.

The invention provides the application of this drug in preparing medicines for treatment of the HPV infection, that is the new application of the human rabies vaccine which is characterized the application in preparing medicines for treatment of the HPV infection therapeutic.

To better realize the purposes of this invention, the human rabies vaccine may be administered by injection, spray, gel or suppository, wherein each dose contains 0.1-15 IU of the human rabies vaccine. To better realize the purposes of this invention, each clinical dose of the human rabies vaccine is preferably 2.5-7.5 IU by Intramuscular injection or membrana mucosa administer.

Compared with the prior art, this invention has the advantages that: the drug disclosed in the invention has the characteristics of safety, effectiveness and no relapses after treatment; the negative-conversion rate in patients with positive HPV-infection reaches 90% after treatment.

### DETAILED DESCRIPTION OF THE INVENTION

This invention can be realized by the following embodiments, free from the limitation of ratio, compositions, methods and steps thereof. It is understood that the embodiments and solutions discussed here are intended for illustration only. Those skilled in this field can come up with various improvements and variations, which shall be included in the spiritual and actual scopes and the scope of the attached claims.

### Example 1

### Production of the Human Rabies Vaccine with the Rabies Strain PV02061

Inoculate the rabies strain PV02061 into the eukaryotic cells (including Vero cells, shrewmouse kidney cells, human diploid cells, chick-embryo cells, preferably the Vero cells), collect the virus liquid, inactivate and purify it, and then add the pharmaceutically acceptable carriers to prepare the human rabies vaccine preparation at 0.5-10.5 IU per dose.

### Example 2

### Production of the Human Rabies Vaccine with the Rabies Strain PM

Inoculate the rabies strain PM into the eukaryotic cells (including Vero cells, shrewmouse kidney cells, human diploid cells, chick-embryo cells, preferably the human diploid cells), collect the virus liquid, inactivate and purify it, and then add the pharmaceutically acceptable carriers to prepare the human rabies vaccine preparation which is 0.5-10.5 IU per dose.

### Example 3

### Production of the Human Rabies Vaccine with the Rabies Strain Vnukovo-32

Inoculate the rabies strain Vnukovo-32 into the eukaryotic cells (including Vero cells, shrewmouse kidney cells, human diploid cells, chick-embryo cells, preferably the shrewmouse kidney cells), collect the virus liquid, inactivate and purify it, and then add the pharmaceutically acceptable carriers to prepare the human rabies vaccine preparation at 0.5-10.5 IU per dose.

### Example 4

### Production of the Human Rabies Vaccine with the Rabies Strain Flury-32

Inoculate the rabies strain Flury-32 into the eukaryotic cells (including Vero cells, shrewmouse kidney cells, human diploid cells, chick-embryo cells, preferably the chick-embryo cells), collect the virus liquid, inactivate and purify it, and then add the pharmaceutically acceptable carriers to prepare the human rabies vaccine preparation at 0.5-10.5 IU per dose.

### Example 5

### Production of the Human Rabies Vaccine with the Rabies Strain aG

Inoculate the rabies strain aG into the eukaryotic cells (including Vero cells, shrewmouse kidney cells, human diploid cells, chick-embryo cells, preferably the chick-embryo cells), collect the virus liquid, inactivate and purify it, and then add the pharmaceutically acceptable carriers to prepare the human rabies vaccine preparation at 0.5-10.5 IU per dose.

### Example 6

### Production of the Human Rabies Vaccine with the Rabies Strain CTN-1

Inoculate the rabies strain CTN-1 into the eukaryotic cells (including Vero cells, shrewmouse kidney cells, human diploid cells, chick-embryo cells, preferably the chick-embryo cells), collect the virus liquid, inactivate and purify it, and then add the pharmaceutically acceptable carriers to prepare the human rabies vaccine preparation at 0.5-10.5 IU per dose.

### Example 7

### Preparation of the Rabies Vaccine Injections

The human rabies vaccines can be prepared as injections. The following preparation schemes are provided to further describe this invention, but they shall not be considered to limit the scope of this invention. Those skilled in this field shall understand that the preparation of the human rabies vaccines varies with excipients, preparation processes, active ingredient contents, filling volumes of the preparations, etc.

Scheme 1: Human Rabies Vaccine Injection without Adjuvant Includes the Following Ingredients Per Dose:

| Human rabies vaccines | 2.5 IU-4.5 IU | |
|---|---|---|
| Disodium hydrogen phosphate•12H₂O | 1.34 | mg |
| Sodium dihydrogen phosphate•2H₂O | 0.20 | mg |
| Sodium chloride | 4.25 | mg |
| Thiomersalate | 0.025 | mg |
| Human albumin | 10 | mg |
| Water for injection | fill up to 0.5 | ml |

Add hydrochloric acid or sodium hydroxide to adjust the pH value to 7.2-8.0 and then subpackage the product to obtain the human rabies vaccine injections.
Scheme 2: Human Rabies Vaccine Freeze-Dried Preparation without Adjuvant Includes the Following Ingredients Per Dose:

| Human rabies vaccines | 2.5 IU-4.5 IU | |
|---|---|---|
| Disodium hydrogen phosphate•12H₂O | 1.34 | mg |
| Sodium dihydrogen phosphate•2H₂O | 0.20 | mg |
| Sodium chloride | 4.25 | mg |
| Dextran | 15 | mg |
| Human albumin | 10 | mg |
| Water for injection | fill up to 0.5 | ml |

Add hydrochloric acid or sodium hydroxide to adjust the pH value to 7.2-8.0, then subpackage and freeze-dry the product to obtain the human rabies vaccine freeze-dried preparations.
Scheme 3: Human Rabies Vaccine Injection with Adjuvant, Wherein the Adjuvant May be PICKCa Adjuvant, Al(OH)₃ Adjuvant or Aluminum Phosphate Adjuvant; the Injection Includes the Following Ingredients Per Dose:

| | | |
|---|---|---|
| Human rabies vaccines: | 1.0 | IU |
| Human rabies vaccines: | 2.5-4.5 | IU |
| Disodium hydrogen phosphate•12H₂O | 1.34 | mg |
| Sodium dihydrogen phosphate•2H₂O | 0.20 | mg |
| Sodium chloride | 4.25 | mg |
| Al(OH)₃ | 0.35 | mg |
| Water for injection | fill up to 0.5 | ml |

Add hydrochloric acid or sodium hydroxide to adjust the pH value to 7.2-8.0 and then subpackage the product to obtain the human rabies vaccine injection.

### Example 8

### Preparation of Human Rabies Vaccine Suppository

The human rabies vaccine can be prepared into suppository. The following preparation scheme is provided to further describe this invention but shall not be considered to limit the scope of this invention. Those skilled in this field shall understand that the preparation of the human rabies vaccines varies with the auxiliary materials, preparation processes, active ingredient contents, suppository weights, etc. Human rabies vaccine suppository per 3 g includes:

| | | |
|---|---|---|
| Human rabies vaccine | 0.5 | IU |
| Gelatin | 0.6 | g |
| Glycerine | 2.1 | g |
| Methylparaben | 5.4 | mg |
| Propyl p-hydroxybenzoate | 0.6 | mg |
| Water for injection | fill up to 3 | g |

### Example 9

### Preparation of the Human Rabies Vaccine Gel

The human rabies vaccine can be prepared as a suppository. The following preparation scheme is provided to further describe this invention but shall not be considered to limit the scope of this invention. Those skilled in this field shall understand that the preparation of the human rabies vaccines varies with the auxiliary materials, preparation processes, active ingredient contents, etc. Human rabies vaccine gel per 10 g includes the following ingredients:

| | | |
|---|---|---|
| Human rabies vaccine | 10.5 | IU |
| Carbomer (934) | 30 | mg |
| Methylparaben | 18 | mg |
| Propyl p-hydroxybenzoate | 2 | mg |
| Glycerol | 1 | g |
| Add the sodium hydroxide to | | |
| adjust the pH to 6-8 | | |
| Water for injection | fill up to 10 | g |

### Example 10

### Preparation of Human Rabies Vaccine Spray

The human rabies vaccine can be prepared as a spray. The following preparation scheme is provided to further describe this invention but shall not be considered to limit the scope of this invention. Those skilled in this field shall understand that the preparation of the human rabies vaccines varies with the auxiliary materials, preparation processes, active ingredient contents, etc.

The human rabies vaccine spray per milliliter includes the following ingredients:

| | | |
|---|---|---|
| Human rabies vaccine | 0.1 | IU |
| Carbomer (934) | 0.3 | mg |
| Methylparaben | 1.8 | mg |
| Propyl p-hydroxybenzoate | 0.2 | mg |
| Glycerol | 0.1 | g |
| Add the sodium hydroxide to | | |
| adjust the pH to 6-8 | | |
| Water for injection | fill up to 1 | ml |

### Example 11

### Treatment of HPV Infection by Administering Human Rabies Vaccine

The curative effects of the human rabies vaccine on HPV infection were studied by preclinical tests on 40 volunteers. All the patients, females aged between 27 and 62, were clinically confirmed to be infected by HPV viruses. By gene testing, the HPV subtypes including HPV6, HPV11, HPV16, HPV18, HPV31, HPV33, HPV35, HPV45, HPV52, HPV56, HPV58, HPV59 and HPV66 were found, and each case was a single subtype infection or multi-subtype cross infection. All volunteers were randomly divided to a test group and a control group; 2.5 IU of human rabies vaccine was administered to the patient in test group by Intramuscular injection once a day; the treatment lasted for 30 days; and some of the patients were under mucomembranous administration with a suppository or spray once a day. The control group was subject to no treatment, and the test group accepted HPV infection detection after two months. The control group was terminated at the first test and accepted the human rabies vaccine treatment after HPV-infection detection; the dosage regimen was the same as previously mentioned, and after two months, the HPV-infection detection was performed. Both the test group and the control group accepted HPV-infection detection after stopping taking the drugs for 1 year.

Those skilled in this field shall understand that the medication method could be local mucocutaneous administration, namely using suppository or spray at a dose of 0.1-10 IU, so that the drugs are absorbed into body via mucous membrane, and that the drugs also can be used for treatment of other subtype HPV infections besides the mentioned subtypes.

The study results showed that: compared with the control group, among the 20 positive HPV virus cases in the control group, 19 turned to be negative after taking the drugs for 1 month. The negative-conversion rate was 95%, while the 20 cases in the control group were still positive. Among the 20 cases in the control group, 18 turned to be negative after taking the drugs for 1 month, and the negative-conversion rate was 90%. After stopping taking drugs for 1 year, among the 40 cases, 35 were still negative. During test, no adverse effects were generated. Table 1 shows a summary of the test groups and detection results.

The study results prove that the human rabies vaccine has obvious curative effects on the HPV infection and generates no side effects or toxic effects.

TABLE 1
Test Group and Detection Results for HPV-infection Treatment
with Human Rabies Vaccine

| | | | HPV Virus Detection Results | | | |
|---|---|---|---|---|---|---|
| | | | Before | | | |
| | | | taking | After taking | 1 year after stopping | |
| Group | | | the drugs | the drugs | taking the drugs | |
| | No. | Age | HPV subtype | | | |
| Test | 1 | 28 | 11 | Negative | Negative | |
| Group | 2 | 30 | 11, 16 | Negative | Negative | |
| | 3 | 42 | 52 | Negative | Negative | |
| | 4 | 35 | 11 | Negative | Negative | |
| | 5 | 40 | 11, 16 | Negative | Negative | |
| | 6 | 41 | 16 | Negative | Negative | |
| | 7 | 35 | 6, 11, 16, | Negative | Negative | |
| | | | 66 | | | |
| | 8 | 45 | 16 | Negative | Negative | |
| | 9 | 27 | 45 | Negative | Negative | |
| | 10 | 25 | 11 | Negative | Negative | |
| | 11 | 33 | 11 | Negative | Negative | |
| | 12 | 46 | 16 | Negative | Negative | |
| | 13 | 30 | 6, 11, 16 | Negative | Negative | |
| | 14 | 38 | 16 | Negative | Negative | |
| | 15 | 40 | 59 | Negative | Negative | |
| | 16 | 39 | 16, 59 | Negative | 56 | |
| | 17 | 62 | 6, 11, 16, | Negative | Negative | |
| | | | 52 | | | |
| | 18 | 28 | 6, 11, 16 | Negative | Negative | |
| | 19 | 56 | 56, 58 | 56, 58 | 56/58 | |
| | 20 | 45 | 18, 31, 58 | Negative | 18/58 | |
| Control | 21 | 32 | 11, 58 | 11 | Negative | Negative |
| Group | 22 | 28 | 16 | 16 | Negative | Negative |
| | 23 | 39 | 31 | 31 | Negative | Negative |
| | 24 | 34 | 16 | 16 | Negative | Negative |
| | 25 | 46 | 33 | 33 | 33 | Negative |
| | 26 | 29 | 11, 16, 66 | 11 | Negative | Negative |
| | 27 | 40 | 11, 16 | 16 | Negative | Negative |
| | 28 | 30 | 16, 39 | 39 | Negative | Negative |
| | 29 | 50 | 59, 66, 58 | 66, 58 | Negative | 58 |
| | 30 | 29 | 16 | 16 | Negative | Negative |
| | 31 | 28 | 16 | 16, 66 | Negative | Negative |
| | 32 | 30 | 16 | 16 | 16 | Negative |
| | 33 | 27 | 16 | 16 | Negative | Negative |
| | 34 | 43 | 11, 16 | 11, 16 | Negative | Negative |
| | 35 | 52 | 6, 11, 16 | 6, 11, 16 | Negative | Negative |
| | 36 | 55 | 6, 11, 16 | 11, 16 | Negative | Negative |
| | 37 | 55 | 35 | 35 | Negative | 35 |
| | 38 | 40 | 11 | 11, 16 | Negative | Negative |
| | 39 | 28 | 58 | 11, 58 | Negative | Negative |
| | 40 | 44 | 16 | 16 | Negative | Negative |

### Example 12

### Animal Acute Toxicity Test

Human rabies vaccines, at about 6,293 times the dose for humans, were injected into to the Kunming mice in single dose. Observation continuously lasting for 14 days after injection showed that all mice were still alive, which suggests the human rabies vaccine is safe.

### Example 13

### Animal Long-Term Toxic Test

The rabbits were administered repetitively by injection of human rabies vaccine for 90 days and observed for two weeks after stopping medication. The detection results of the routine blood tests, the biochemical indicators and the dissection results suggested that the human rabies vaccine is safe.

### Example 14

### Local Irritation Test

Local irritation from the human rabies vaccine spray was studied by local administration. The drugs were applied to the vaginas of the female rabbits for 30 consecutive days to observe the local irritation. The results suggested that the human rabies vaccine spray is nonirritant.

1-6. (canceled)

7. A method of treating Human Papilloma Virus infection in a human subject suffering therefrom comprising administering to the human subject a pharmaceutical composition comprising a human rabies vaccine prepared from any one of the rabies vaccine selected from the group consisting of PV-2061, PM, Vnukovo-32, Flurry, aG, CTN-1 and CVS and at least one pharmaceutically acceptable carrier.

8. The method of claim 7 wherein the pharmaceutical composition is administered by injection.

9. The method of claim 8 wherein the injection is intramuscular.

10. The method of claim 9 wherein the pharmaceutical composition administered to the human subject contains a dose of 2.5 to 7.5 IU of the human rabies vaccine.

11. The method of claim 7 wherein the pharmaceutical composition is administered to the mucosal membrane of the human subject.

12. The method of claim 11 wherein the pharmaceutical composition is administered as a spray, gel or suppository.

13. The method of claim 11 wherein the wherein the pharmaceutical composition administered to the human subject contains a dose of 2.5 to 7.5 IU of the human rabies vaccine.

14. The method of claim 7 wherein the pharmaceutical composition administered to the human subject contains a dose of 0.1 to 15 IU of human rabies vaccine.

15. The method of claim of claim 7 wherein the pharmaceutical composition consists of the human rabies vaccine and at least one pharmaceutically acceptable carrier.
